(19) United States
(12) Patent Application Publication
(10) Pub. No.: US 2026/0295263 A1
(43) Pub. Date: Oct. 1, 2026

(54) BAROREFLEX ACTIVATION THERAPY FOR THE TREATMENT OF SECONDARY PULMONARY HYPERTENSION

(71) Applicant: CVRx, Inc., Minneapolis, MN (US)

(72) Inventors: Philip B. Adamson, Austin, TX (US); Seth J. Wilks, Valparaiso, IN (US); Paul Pignato, Jacksonville, FL (US); Tucker Stuart, North Saint Paul, MN (US); Bart Carey, Maplewood, MN (US)

(21) Appl. No.: 19/552,227

(22) Filed: Feb. 27, 2026

Related U.S. Application Data
(60) Provisional application No. 63/780,990, filed on Mar. 31, 2025.

(51) Int. Cl. A61N 1/36

(52) U.S. Cl. CPC A61N 1/36117; A61N 1/36031; A61N 1/36034; A61N 1/36139; A61N 1/3615

(57) ABSTRACT

This disclosure presents systems and methods for treating secondary pulmonary hypertension (PH) using baroreflex activation therapy (BAT). A stimulation device delivers electrical stimulation to a baroreceptor to modulate autonomic nervous system activity and reduce pulmonary vascular resistance and right ventricular afterload. Pulmonary arterial pressure is measured using one or more pressure sensors, and stimulation parameters are automatically adjusted based on the measured pressure. In certain embodiments, a closed-loop control algorithm regulates stimulation intensity using pulmonary arterial pressure and additional physiological parameters, including right atrial or systemic arterial pressure.

Therapy parameters may be adjusted during rest and exercise states and selected based on a dose-response relationship between stimulation intensity and pulmonary pressure reduction. Historical pulmonary pressure data may be used to predict future pressure trends and refine therapy delivery. These adaptive systems provide physiologically guided management of secondary PH through autonomic modulation.

### CLAIM OF PRIORITY

This non-provisional patent document claims the benefit of priority under 35 U.S.C. § 119(e) to U.S. Provisional Patent Application Ser. No. 63/780,990, entitled “BAROREFLEX ACTIVATION THERAPY FOR THE TREATMENT OF SECONDARY PULMONARY HYPERTENSION,” filed on Mar. 31, 2025, which is incorporated herein by reference in its entirety.

### TECHNICAL FIELD

This patent document relates to medical therapies for cardiovascular disorders, specifically addressing secondary pulmonary hypertension (PH).

### BACKGROUND

Secondary PH, also known as World Health Organization (WHO) Group 2 PH, is a progressive and life-threatening condition characterized by elevated pulmonary artery pressure secondary to left ventricular systolic or diastolic dysfunction or left-sided valvular disease. WHO Group 2 PH is defined by a mean pulmonary artery pressure greater than 20 mmHg, and pressures exceeding this threshold are associated with increased mortality rates due to their detrimental effects on right ventricular function and overall cardiovascular stability.

Among patients with symptomatic heart failure classified as New York Heart Association (NYHA) Class II, III, or IV, up to 82% are at risk of developing secondary PH due to elevated left-sided filling pressures leading to increased pulmonary artery pressure. Secondary PH arises from decreased pulmonary artery compliance, which leads to increased pulmonary vascular resistance in response to left ventricular dysfunction. These pathophysiological changes collectively contribute to progressive right heart strain and pulmonary congestion. As the heart struggles to pump blood effectively through the pulmonary circulation, right ventricular failure progressively worsens, significantly limiting both quality of life and life expectancy.

Current therapeutic strategies for secondary PH primarily focus on managing the underlying cause of the disease. Guideline-recommended medical therapies serve as the primary pharmacologic interventions to treat underlying heart disease; however, as left ventricular dysfunction worsens, treatment may also require mechanical circulatory support or cardiac transplantation. Adjunctive drug treatments specifically targeting secondary PH remain limited, representing a significant treatment gap. Secondary PH treatments primarily consist of vasodilators, which may pose significant clinical risks for some patients. While these interventions may provide symptomatic relief, they do not directly address the autonomic dysfunction that contributes to disease progression.

### OVERVIEW

The present inventors recognize baroreflex activation therapy (BAT) as a targeted therapeutic approach for mitigating the pathophysiological mechanisms underlying secondary PH. BAT involves controlled electrical stimulation of baroreceptors, such as those located in the carotid sinus, to modulate autonomic nervous system activity by reducing sympathetic nervous system activity and enhancing parasympathetic tone. This autonomic modulation has been shown to lower pulmonary vascular resistance, improve cardiac function, and provide clinical benefits in conditions such as resistant hypertension and heart failure.

Given the significant role of sympathetic overactivity in secondary PH, BAT serves as a promising intervention to reduce pulmonary vascular resistance, improve hemodynamic stability, and slow disease progression. Pulmonary artery pressure measurements, obtained through either an invasive pressure sensor—such as Abbott's CARDIOMEMS sensor or Edwards Lifesciences' CORDELLA sensor—or a non-invasive pressure sensor, such as a wearable or subcutaneous sensor, may be integrated into a treatment protocol to enable dynamic monitoring and titration of BAT. This continuous or periodic assessment of pulmonary hemodynamics allows for therapy adjustments, ensuring that BAT remains responsive to physiological changes and optimally tailored to the patient's evolving condition. Additionally, or alternatively, right atrial, and pulmonary artery pressure measurements obtained via right heart catheterization can provide data for therapy adjustments within a closed-loop or open-loop control system.

The disclosed systems and methods provide an innovative and adaptable means for treating secondary PH through BAT. By leveraging BAT's capacity to modulate autonomic nervous system activity, these systems and methods are designed to reduce pulmonary vascular resistance, alleviate right ventricular strain, and enhance overall cardiovascular function in patients with secondary PH.

The system comprises an implantable or external BAT device designed to deliver stimulation—including electrical, mechanical, chemical, optical, or other forms—to baroreceptors, such as those located within the carotid sinus. This stimulation reduces sympathetic nervous system activity while simultaneously increasing vagal efferent activity, leading to enhanced vasodilation within the pulmonary circulation. The resulting decrease in pulmonary artery pressure alleviates right ventricular afterload, promotes hemodynamic stability, and ultimately improves patient outcomes by mitigating the progression of secondary PH.

The system can integrate pressure monitoring technologies to enable therapy adjustments. Pulmonary artery pressure can be measured using invasive pressure sensors as well as through right heart catheterization and pressure-volume loop analysis. These measurements can be incorporated within a dynamic closed-loop or open-loop control system to optimize BAT stimulation parameters based on individualized patient needs. Additionally, non-invasive devices capable of estimating pulmonary pressures may also be utilized within a closed-loop or open-loop BAT stimulation strategy, providing an alternative approach to invasive pressure sensors.

The disclosed systems and methods are applicable to both acute and chronic cases of secondary PH, providing an adaptable treatment strategy that can be customized based on physiological data. Furthermore, the system can incorporate a dose-response feedback model, which allows for correlation of sensor-derived pulmonary artery pressure data with BAT stimulation parameters to assess and optimize therapy effectiveness on a personalized basis.

Beyond primary hemodynamic improvements, BAT therapy for secondary PH offers additional benefits, including reduced stress and anxiety associated with hypertensive events, particularly during times of physical activity or exertion. These additional benefits may be evaluated through patient-reported outcomes using standardized questionnaires, enabling a holistic assessment of therapy impact.

In summary, the present systems and methods introduce a novel application of BAT for the treatment of secondary PH, integrating autonomic modulation with physiological feedback to offer a comprehensive, adaptable, and patient-centered therapeutic approach.

These and other examples and features of the disclosed systems and methods will be described in greater detail in the following Detailed Description. This Overview is intended to present non-limiting examples of the disclosed subject matter and is not meant to serve as an exhaustive or exclusive explanation. Instead, the Detailed Description below provides further information regarding the design, implementation, and applications of the disclosed systems and methods.

### BRIEF DESCRIPTION OF THE DRAWINGS

In the drawings, like numerals are used to describe similar features and components throughout several views. The drawings illustrate generally, by way of example, but not by way of limitation, various system and method embodiments discussed in this patent document.

FIG. 1 illustrates a schematic representation of a BAT system, including an implantable pulse generator (IPG), an electrode or electrode array positioned on or near the carotid sinus, an external programmer, a patient interface, and various sensor options, as constructed in accordance with at least one embodiment.

FIG. 2 depicts an implantation process, showing the placement of the electrode or electrode array near the carotid sinus to achieve targeted baroreceptor engagement, as constructed in accordance with at least one embodiment.

FIGS. 3A-B illustrate implantable pressure sensors, such as Abbott's CARDIOMEMS sensor and Edwards Lifesciences' CORDELLA sensor, which can be integrated into the BAT system for continuous pulmonary artery pressure monitoring, as constructed in accordance with at least one embodiment.

FIG. 3C shows an alternative right heart catheterization approach, providing direct pulmonary pressure measurement via a catheter inserted through the jugular, brachial, or femoral vein, as constructed in accordance with at least one embodiment.

FIG. 4 provides a detailed schematic of an adaptive closed-loop control system within the BAT system. This diagram illustrates how sensor feedback, control algorithms, and a feedback loop can dynamically regulate stimulation intensity to optimize pulmonary hemodynamics, as constructed in accordance with at least one embodiment.

FIG. 5 demonstrates an intermittent pressure monitoring system that enhances therapy adaptation by reducing sensor fatigue. The system integrates physiological monitoring, enabling continuous therapy optimization while maintaining sensor efficiency, as constructed in accordance with at least one embodiment.

FIG. 6 presents a comprehensive monitoring framework for BAT therapy, integrating patient-reported outcomes, sensor-based physiological assessments, and clinical evaluation tools, as constructed in accordance with at least one embodiment.

FIG. 7 illustrates a dose-response analysis model, correlating pulmonary artery pressure reduction with BAT stimulation intensity. This model can enable determination of optimal therapy settings, ensuring maximal therapeutic benefit while preventing overstimulation, as constructed in accordance with at least one embodiment.

The drawings are not necessarily to scale. Certain features and components may be shown exaggerated in scale or in schematic form, and some details may not be shown in the interest of clarity and conciseness.

### DETAILED DESCRIPTION

The present systems and methods provide a means for utilizing BAT in the treatment of secondary PH. The systems and methods are designed to modulate autonomic nervous system activity, leading to a reduction in pulmonary vascular resistance, improved hemodynamic function, and enhanced quality of life for patients with secondary PH. By integrating physiological monitoring, feedback control, and adaptive therapy titration, the disclosed systems and methods can optimize BAT stimulation to effectively address the hemodynamic burden associated with secondary PH.

### Pathophysiology of Secondary PH

Secondary PH is a progressive and life-threatening disorder primarily caused by chronic left ventricular failure, leading to pulmonary venous congestion, vascular remodeling, and increased pulmonary vascular resistance (PVR). As pulmonary artery pressure elevates over time, the resultant increase in right ventricular (RV) afterload contributes to RV dysfunction, ultimately leading to higher morbidity and mortality rates.

The early stages of secondary PH are characterized by passive congestion in the pulmonary veins, which elevates capillary hydrostatic pressure and triggers endothelial cell stress and vascular inflammation. As the disease progresses, intimal thickening, medial hypertrophy, and perivascular fibrosis develop, leading to further increases in PVR and exacerbation of right ventricular afterload.

In addition to hemodynamic alterations, sympathetic nervous system overactivity plays a critical role in sustaining pulmonary vasoconstriction, worsening endothelial dysfunction, and exacerbating neurohormonal activation.

These maladaptive responses worsen pulmonary arterial hypertension and contribute to progressive right heart strain, limiting both cardiac output and overall circulatory efficiency.

This pathophysiological cascade underscores the need for a therapy that directly addresses autonomic dysfunction, rather than simply managing the symptoms of secondary PH.

### Role of Baroreflex Activation Therapy in Secondary PH Management

Conventional treatments for secondary PH, including diuretics and pulmonary vasodilators, primarily provide symptomatic relief without addressing the underlying autonomic dysregulation that contributes to disease progression.

While these treatments help manage the symptoms, they do not target the fundamental mechanisms driving pulmonary vascular dysfunction and right ventricular strain. BAT offers a mechanistic intervention that directly modulates baroreceptor activity, restoring autonomic balance, attenuating sympathetic nervous system overactivity, and enhancing both systemic and pulmonary vascular compliance to improve long-term outcomes.

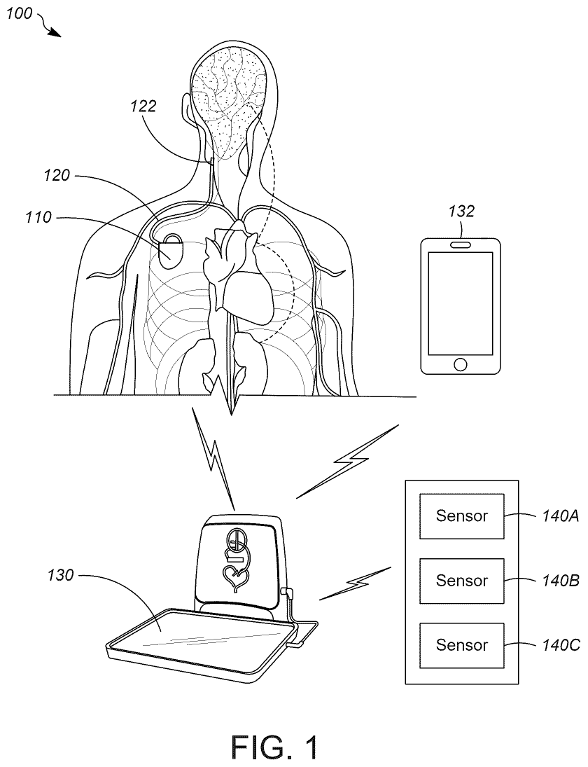

As illustrated in the example of FIG. 1, the BAT system (100) comprises several components, including an IPG (110), an electrode or electrode array (122) positioned on or near the carotid sinus and connected to the IPG by a flexible lead (120), an external programmer (130), a patient interface (132), and various sensor (140A, 140B, 140C) options, examples of which are further detailed below. This system (100) delivers controlled baroreceptor stimulation, which reduces sympathetic outflow while augmenting parasympathetic activity. The resulting autonomic modulation leads to a decrease in mean pulmonary artery pressure, alleviation of right ventricular afterload, and improved ventricular-vascular coupling. Additionally, the therapy can be titrated based on pressure sensor feedback, allowing for adjustments that optimize hemodynamic stability and enhance therapeutic efficacy.

By directly counteracting autonomic dysfunction, BAT provides a targeted, long-term therapeutic approach that complements existing pharmacologic treatments while addressing the pathophysiological drivers of secondary PH. This intervention has the potential to improve both acute and chronic clinical outcomes by mitigating pulmonary vascular resistance, enhancing cardiac efficiency, and reducing the progression of right heart failure. Through its mechanistic approach, BAT represents a novel and adaptive treatment strategy that integrates neuromodulation, hemodynamic monitoring, and personalized therapy adjustments to enhance patient outcomes in secondary PH management.

### IPG Stimulation Parameters

The IPG (110) is designed with stimulation parameters-including pulse amplitude, pulse frequency, and pulse duration-that can be dynamically adjusted based on physiological feedback. This optionally closed-loop system automatically modulates stimulation intensity in response to hemodynamic signals, such as pulmonary artery pressure or right atrial pressure, ensuring that therapy delivery remains precise and adaptive. By integrating continuous monitoring with stimulation adjustments, the system (100) can optimize hemodynamic stability while minimizing the need for manual recalibration, thereby enhancing both treatment efficacy and patient safety.

To ensure long-term functionality, the BAT system (100) can incorporate energy-efficient design features, including advanced battery technology, low-power communication protocols, and predictive stimulation algorithms. These innovations help extend the lifespan of the components while maintaining consistent and effective therapy delivery. The ability to fine-tune stimulation in response to changing physiological conditions ensures that patients receive optimal therapy without frequent interventions.

The integration of sensor-based (140A, 140B, 140C) closed-loop control and adaptive titration positions BAT as a mechanistically targeted, long-term intervention for secondary PH. By directly addressing autonomic dysfunction, this approach can provide a complementary therapy to existing pharmacologic and device-based treatments. Through its intelligent adaptation to the patient's evolving condition, the BAT system (100) can offer a highly personalized and responsive therapeutic strategy, improving both short-term and long-term clinical outcomes.

### Signal Processing and Therapy Customization

The IPG (110), external programmer (130), and/or patient interface (132) can utilize advanced signal processing algorithms to continuously analyze sensor (140A, 140B, 140C) feedback. By monitoring fluctuations in pulmonary artery pressure, the system (100) dynamically optimizes stimulation intensity to ensure precise and effective autonomic modulation. This continuous adjustment allows the therapy to remain responsive to the patient's evolving hemodynamic profile, improving overall treatment effectiveness while reducing the need for manual recalibration.

To further enhance therapy customization, the control algorithm can integrate sensor fusion techniques, combining data from multiple physiological inputs. These can include heart rate variability, activity level, respiratory cycle dynamics, and autonomic biomarkers, all of which contribute to refining the titration of stimulation parameters. By leveraging this multi-source feedback, the system (100) improves the accuracy of therapy adjustments, ensuring that each patient receives an adaptive and individualized treatment tailored to their specific physiological needs. This intelligent signal processing approach enhances the precision of baroreceptor stimulation, ultimately optimizing patient outcomes in the management of secondary PH.

### External Programmer and Clinician Interface

The external programmer (130) serves as a central interface for clinicians, enabling therapy monitoring and precise adjustments to patient treatment. This programmer provides a streamlined platform through which healthcare professionals can assess the effectiveness of BAT and modify stimulation settings as needed to optimize patient outcomes. By offering direct control over therapy parameters, the external programmer (130) ensures that BAT remains adaptable and responsive to each patient's evolving condition, allowing for dynamic, data-driven adjustments that enhance therapeutic effectiveness.

A feature of the clinician interface is its advanced programming screen, designed with protected access to prevent unauthorized modifications. This safeguard ensures the integrity and security of therapy settings, reducing the risk of accidental or improper alterations. Additionally, the programmer (130) can incorporate expanded security and usability features that enhance patient safety while streamlining clinical workflows. To support data-driven decision-making, a high-level dashboard can aggregate patient data, which can optionally utilize AI-driven analytics to generate a comprehensive patient health score. This data visualization feature enables clinicians to quickly assess patient status and make informed therapeutic decisions based on objective physiological trends.

To further enhance therapy optimization, the external programmer (130), or alternatively the IPG (110), can be integrated with a data storage module, which is configured to record pulmonary pressure measurements alongside corresponding baroreflex activation therapy settings. This data archiving capability allows clinicians to review historical pressure data, assess therapy effectiveness over time, and refine stimulation protocols based on patient-specific responses. By maintaining a comprehensive record of hemodynamic trends and therapy adjustments, the data storage module supports long-term patient management, aiding in treatment planning, predictive analytics, and regulatory compliance.

Additionally, the external programmer (130) can feature an alert system that notifies clinicians of significant changes in patient hemodynamics. This function guides clinicians to a detailed evaluation and therapy adjustment screen, where they can analyze patient responses and implement appropriate modifications to stimulation parameters. By integrating these advanced monitoring, data storage, and adjustment capabilities, the external programmer (130) enhances the precision, safety, and efficiency of baroreflex activation therapy, ultimately improving patient care and long-term clinical outcomes.

### Patient User Interface

The BAT system (100) incorporates a dedicated patient interface (132 to facilitate seamless communication between patients and healthcare providers.

Through this interface, patients can report symptoms, treatment preferences, or concerns, which are then transmitted to a central office (e.g., the external programmer (130)) for analysis. This remote communication capability enables clinicians to assess patient status and make therapy adjustments without requiring in-person visits, ensuring that treatment remains continuous, adaptive, and responsive to the patient's evolving condition.

While the system (100) may allow patients to initiate therapy updates on their own, such as by pressing a designated button (e.g., a green button) on the dedicated patient interface (132) to trigger the download of a new titration program, certain therapy modifications may require clinician oversight. To ensure patient safety and optimal treatment outcomes, some adjustments-particularly those involving significant changes to stimulation parameters-may be subject to approval by a healthcare provider before implementation. In such cases, the system (100) can be configured to notify a clinician, who can then review the request, assess relevant patient data, and authorize or modify the therapy update as needed. To further enhance usability, the patient interface (132) can be designed to be intuitive and user-friendly, incorporating simple feedback mechanisms that allow patients to easily communicate their therapy preferences. Patients can select a smiley face button to maintain their current therapy settings or a sad face button to indicate dissatisfaction with their current therapy, prompting a review for potential modification. However, automatic adjustments in response to patient input may also be subject to predefined safety limits or require clinician confirmation, ensuring that therapy changes remain within safe and effective parameters.

Each patient has access only to their designated screen, providing a secure, personalized experience tailored to their specific treatment requirements. This customized approach enhances patient engagement, improves adherence to therapy, and ultimately contributes to better treatment outcomes while maintaining an appropriate level of clinician oversight to ensure safety and efficacy.

### IPG and Lead Placement for Targeted Baroreceptor Engagement

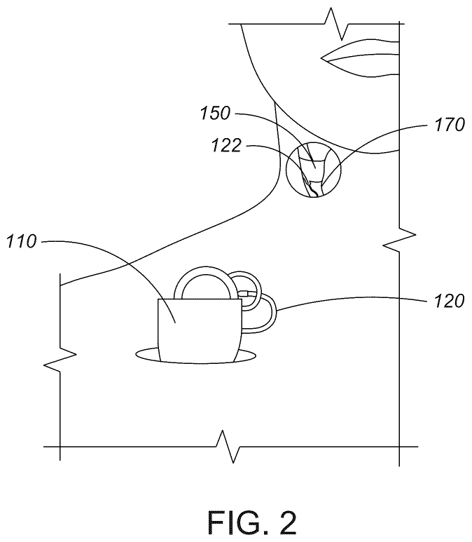

FIG. 2 illustrates the implantation process, highlighting the strategic placement of the electrode or electrode array (122) near the carotid sinus (150) and carotid artery (170) to achieve precise baroreceptor engagement. This targeted placement is essential for effective autonomic modulation, as it enables the system to directly influence the baroreflex pathway, which plays a critical role in cardiovascular regulation. By stimulating the baroreceptors, the BAT system promotes pulmonary vasodilation, helping to reduce pulmonary vascular resistance and alleviate the strain on the right ventricle.

The precise positioning of the electrode(s) (122) ensures optimal therapy delivery, allowing for sustained modulation of sympathetic and parasympathetic activity. This mechanism not only enhances hemodynamic stability but also contributes to improved right ventricular efficiency, ultimately reducing the progression of pulmonary hypertension-related complications.

### Pressure Monitoring and Control Mechanism

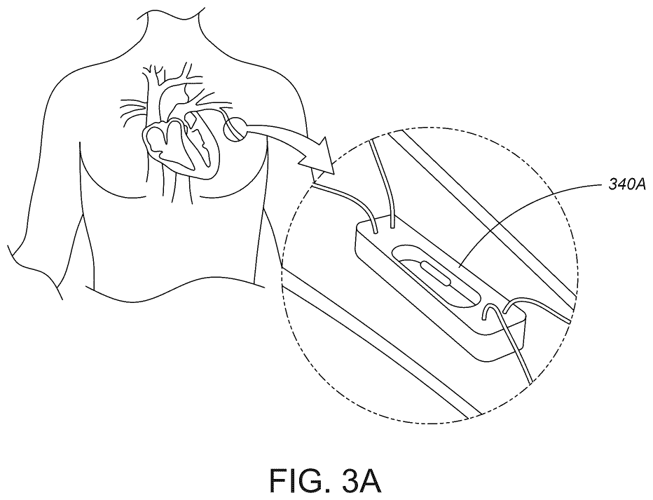

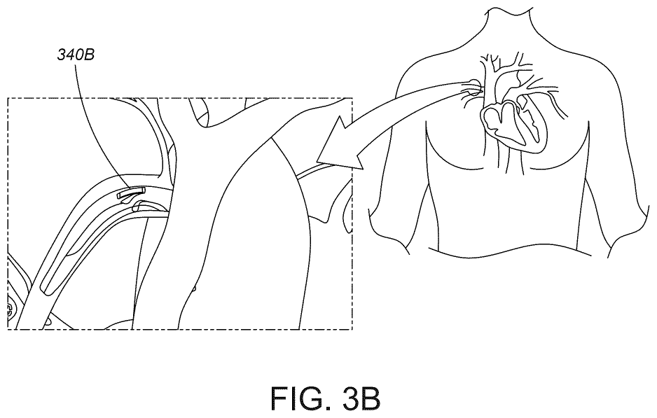

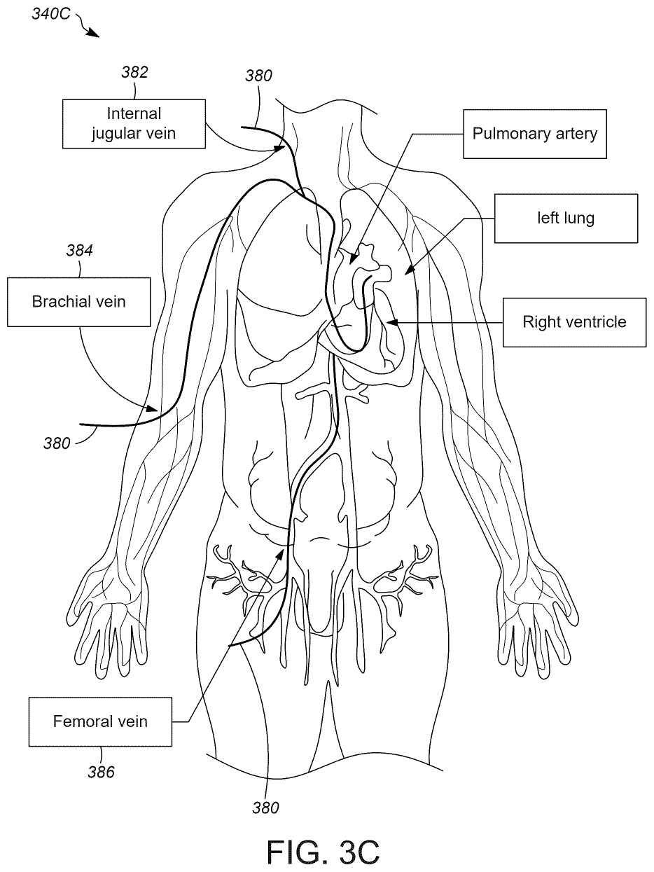

To enable precise and responsive therapy adjustments, the BAT system can integrate pulmonary artery pressure monitoring using implantable pressure sensors. Examples of such sensors include Abbott's CARDIOMEMS sensor (340A) and Edwards Lifesciences' CORDELLA sensor (340B), as illustrated in FIGS. 3A and 3B, respectively. These sensors (340A, 340B) can provide continuous hemodynamic data, allowing for individualized therapy titration based on physiological feedback. By dynamically adjusting baroreceptor stimulation in response to pulmonary artery pressure fluctuations, the system enhances autonomic modulation, effectively addressing the hemodynamic burden of secondary PH and improving overall cardiovascular stability.

In addition to implantable sensors, FIG. 3C illustrates right heart catheterization (340C) as an alternative modality for pulmonary pressure measurement. This technique involves the introduction of a catheter (380) via the jugular (382), brachial (384), or femoral (386) vein, enabling precise, direct measurement of pulmonary artery pressure. Right heart catheterization (340C) can provide a comprehensive hemodynamic profile, which can be useful for both baseline assessment and ongoing therapy adjustments during BAT treatment. By incorporating these advanced pressure monitoring mechanisms, the BAT system ensures that therapy remains aligned with the patient's evolving cardiovascular status, optimizing treatment efficacy and long-term patient outcomes.

### Loop Control and Adaptive Feedback

The BAT system can dynamically modulate stimulation intensity in response to sensor feedback, ensuring precise and continuous optimization of therapy. By incorporating both closed-loop and open-loop configurations, the system provides flexible treatment customization, allowing adjustments to be tailored to patient-specific hemodynamic profiles for secondary PH management.

In closed-loop mode, BAT stimulation intensity is automatically adjusted based on pulmonary artery pressure fluctuations, allowing the therapy to adapt in real time to the patient's evolving hemodynamic status. This mode utilizes sensor-driven feedback to maintain optimal autonomic modulation, improving pulmonary vascular compliance and enhancing overall cardiovascular stability. By continuously monitoring physiological changes, the closed-loop system ensures that therapy remains responsive and effective without requiring manual intervention, making it a highly adaptive and automated therapeutic approach.

In open-loop mode, therapy adjustments are manually controlled by either the clinician or the patient using, for example, preset pressure thresholds. This approach allows for individualized fine-tuning of stimulation settings based on clinical assessments, clinician-directed titration strategies, or the patient's qualitative status. Open-loop control provides greater flexibility for cases where real-time physiological feedback may not be necessary, or when patient-specific conditions require a customized therapy approach. By offering both closed-and open-loop options, the BAT system maximizes adaptability, ensuring that therapy remains tailored to each patient's unique needs.

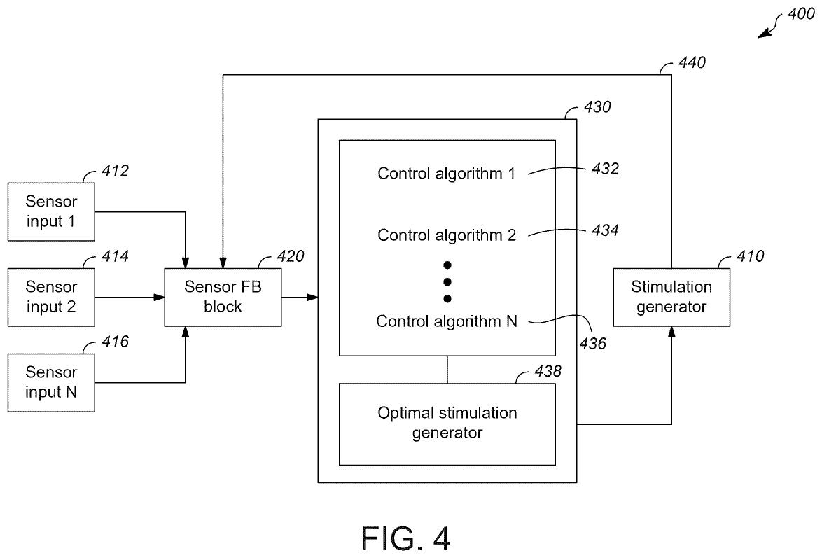

FIG. 4 illustrates an adaptive closed-loop control system (400) designed to optimize pulmonary hemodynamics through real-time sensor feedback and dynamic regulation of stimulation output.

The system begins with sensor inputs (412, 414, 416), which detect key physiological parameters such as pulmonary artery pressure, blood flow, and right atrial pressure. These inputs can lead to a sensor feedback block (420), where the collected data is processed before being transmitted to the control system.

Next, the sensor feedback block (420) can relay the captured physiological data to the control algorithms (432, 434, 436). This block is depicted with an arrow showing the flow of information from the sensors to a computational processing unit (430). The control algorithms (432, 434, 436) analyze the sensor data in real time, computing optimal stimulation parameters (438) tailored to the patient's physiological state. The figure shows an arrow from the computational processing unit (430) leading to the stimulation generator 410 (e.g., the IPG), which delivers precisely modulated electrical stimulation to the BAT system.

To ensure adaptive control, a feedback loop (440) is incorporated, connecting the stimulation generator (410) back to the sensor feedback block (420). This continuous monitoring allows for adjustments in stimulation intensity based on real-time physiological responses, ensuring dynamic regulation and optimization of pulmonary hemodynamics across various activity states, including rest, exercise, and ambulatory movement.

By integrating these elements—sensor detection, feedback processing, algorithmic control, stimulation delivery, and real-time adjustment—the system (400) enables a responsive and individualized therapy approach to enhance cardiovascular function.

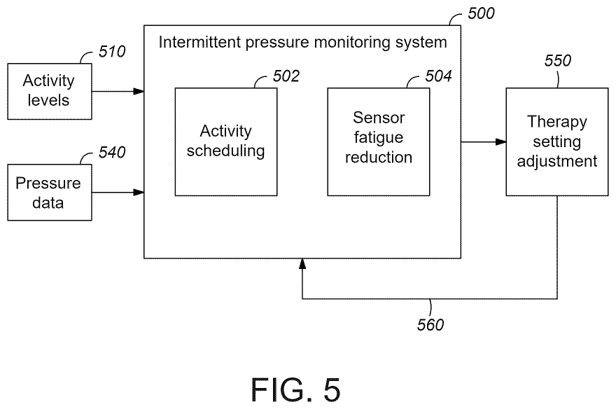

FIG. 5 illustrates an intermittent pressure monitoring system (500), which can enhance therapy adaptation by dynamically adjusting monitoring schedules based on patient activity levels while reducing sensor fatigue.

At the core of the figure is a block representing the intermittent pressure monitoring system (500), which receives inputs including activity levels (510) and pressure data (540), as shown by arrows leading into the block. Within this system (500), key processes can include activity scheduling (502), which can adapt monitoring frequency to different physiological states (e.g., rest, exercise, or ambulatory movement), and sensor fatigue reduction (504), which can ensure prolonged sensor longevity by minimizing unnecessary continuous monitoring.

Arrows extend from the intermittent pressure monitoring system (500) to a therapy settings adjustment (550) block, which can, for example, be integrated with the IPG or external programmer, signifying the system's role in dynamically refining therapy parameters based on real-time data. A feedback loop (560) is incorporated, returning data from the therapy settings adjustment (550) block back to the monitoring system (500), ensuring continuous optimization based on evolving pulmonary pressure dynamics.

By integrating intermittent monitoring with adaptive feedback mechanisms, the BAT system ensures therapy remains responsive to dynamic pulmonary pressure changes. This precision-based approach optimizes treatment effectiveness, reduces right ventricular workload, and enhances long-term patient outcomes in managing secondary pulmonary hypertension.

### Patient Selection and Clinical Implementation

BAT is suitable for both acute and chronic cases of secondary PH, particularly in patients with left ventricular failure and a mean pulmonary artery pressure exceeding 20 mmHg. Careful patient selection is crucial to ensuring optimal therapeutic effectiveness, as BAT is most beneficial for individuals with elevated pulmonary vascular resistance and persistent pulmonary hypertension despite receiving optimal medical therapy. Identifying the right candidates for BAT enhances its ability to improve autonomic regulation, reduce pulmonary vascular tone, and enhance overall cardiovascular function.

Patients eligible for BAT may undergo comprehensive clinical assessments to evaluate their pulmonary hemodynamics and ventricular function. Key diagnostic evaluations may include echocardiography, right heart catheterization, and pressure-volume loop analysis. Echocardiography assesses ventricular function, pulmonary pressures, and right ventricular performance, providing a non-invasive method for evaluating cardiopulmonary health. Right heart catheterization directly measures pulmonary artery pressure, right atrial pressure, and cardiac output, offering precise hemodynamic data that guides treatment decisions. Pressure-volume loop analysis, evaluates ventricular-vascular coupling and quantifies pulmonary vascular resistance, helping clinicians determine the severity of pulmonary hypertension and assess BAT's potential benefits.

By identifying patients with persistent pulmonary hypertension that remains unresponsive to conventional medical management, BAT can be introduced as a complementary intervention. Through autonomic modulation, BAT helps restore cardiovascular balance, reduce pulmonary arterial resistance, and improve hemodynamic stability, making it a promising therapeutic option for carefully selected secondary PH patients.

### Therapeutic Benefits and Additional Outcomes

The primary objective of BAT in the management of secondary PH is to achieve sustained reductions in pulmonary vascular resistance while enhancing right ventricular function through precise autonomic modulation. By continuously adjusting stimulation parameters in response to pulmonary pressure monitoring, BAT provides long-term hemodynamic improvements while minimizing patient burden. This adaptive therapy optimization ensures that BAT remains effective and responsive even as disease progression or physiological conditions change over time.

Beyond its primary hemodynamic benefits, BAT also provides several additional advantages that contribute to improved patient quality of life. One significant benefit is the reduction of stress and anxiety associated with hypertensive events, particularly during physical exertion. By stabilizing pulmonary pressures and reducing sympathetic overactivity, BAT helps patients experience fewer hypertensive episodes, leading to greater emotional well-being and improved overall health. Additionally, BAT enhances exercise tolerance, allowing patients to increase physical activity levels and regain functional capacity. This improvement in physical endurance translates to better mobility, enhanced independence, and a higher quality of life for individuals affected by secondary PH.

By addressing both hemodynamic and quality-of-life outcomes, BAT represents a comprehensive therapeutic approach for secondary PH management, offering long-term stability, improved cardiovascular function, and enhanced patient well-being.

### Comprehensive Monitoring and Adaptive Therapy Titration

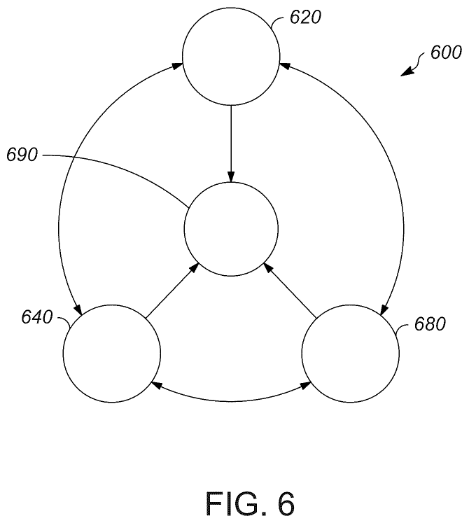

The comprehensive monitoring framework for BAT can integrate multiple assessment modalities to provide a holistic evaluation of therapy effectiveness beyond hemodynamic parameters. FIG. 6 illustrates this multifaceted framework (600) for assessing patient response to BAT therapy, integrating three key components: patient-reported outcomes (620), sensor-based physiological assessments (640), and clinical evaluation tools (680). These components are arranged in a circular layout, emphasizing their interconnectivity and collective contribution to evaluating BAT's impact on secondary pulmonary hypertension management. Patient-reported outcomes (620) can include, for example, quality of life surveys and symptom reporting, providing subjective insights into treatment effects on daily function and well-being. Sensor-based physiological assessments (640) can include, for example, hemodynamic monitoring, pulmonary artery pressure measurements, and activity tracking, offering real-time objective data on cardiovascular function and response to therapy. Clinical evaluation tools (680) can encompass, for example, routine clinical check-ups, imaging, and biomarker analysis, ensuring comprehensive medical assessments of BAT therapy's physiological impact.

Arrows illustrate data flow between these three components, demonstrating how insights from one domain influence refinements in others. The multifaceted framework (600) can be integrated into system feedback loops, ensuring that clinical interventions are guided by both subjective patient experiences and objective physiological data.

At the center of the framework (600), an integration/analysis node (690) serves as the convergence point for these data streams. This node synthesizes inputs from all three assessment domains, facilitating a comprehensive, data-driven approach to optimizing BAT therapy. By integrating subjective and objective measures within a unified assessment framework (600), this approach ensures a holistic evaluation of BAT therapy's effectiveness in managing secondary pulmonary hypertension, enabling personalized treatment adjustments for improved patient outcomes.

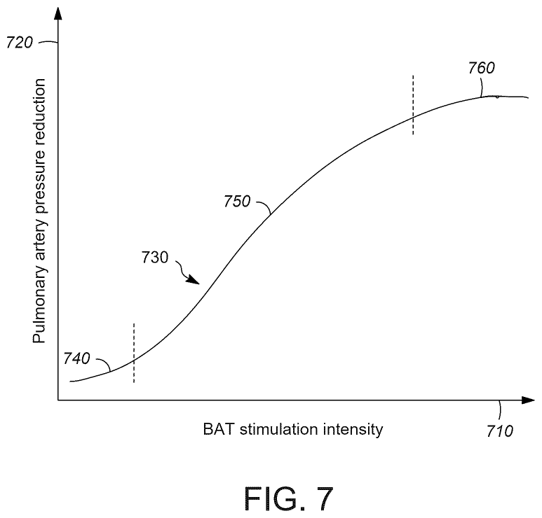

FIG. 7 presents a dose-response analysis model illustrating the relationship between pulmonary artery pressure reduction and BAT stimulation intensity. This model provides a structured framework for optimizing therapy settings, ensuring that each patient achieves maximal therapeutic benefit while avoiding overstimulation. The figure features a sigmoidal (S-shaped) curve, representing the dose-response relationship, with key regions highlighted to guide optimal therapy titration.

The X-axis (710) is labeled “BAT Stimulation Intensity,” showing increasing stimulation levels from left to right, with a gradient scale representing the range from low to high intensity. The Y-axis (720) is labeled “Pulmonary Artery Pressure Reduction,” with a scale that depicts incremental reductions in pressure from bottom to top-indicating lower reductions near the bottom and greater pressure reductions higher on the axis. Alternatively, the X-axis and Y-axis labels and orientations may be reversed, depending on the desired data presentation format.

The dose-response curve (730) follows a sigmoidal trajectory, with three distinct phases: An initial phase (lower left segment), a linear phase (middle segment), and a plateau phase (upper right segment). In the initial phase (lower left segment), with low levels of BAT stimulation intensity, pulmonary artery pressure reduction is minimal, represented by the relatively flat portion of the curve. This suggests that insufficient stimulation (740) does not generate a significant hemodynamic response.

In the linear phase (middle segment), as BAT stimulation intensity increases, pulmonary artery pressure reduction improves significantly. This is the optimal therapy range (750), where effective BAT activation leads to a meaningful reduction in pressure with efficient energy utilization.

In the plateau phase (upper right segment), at higher levels of BAT stimulation intensity, additional reductions in pulmonary artery pressure begin to plateau, indicating diminishing therapeutic returns. This area includes the overstimulation zone (760), where excessive BAT activation may not only fail to provide additional benefit but could also introduce unwanted side effects or inefficiencies.

By integrating physiological monitoring, therapy titration, and patient-centered outcome tracking, this approach ensures a comprehensive and adaptable solution for managing secondary pulmonary hypertension. Through continuous optimization of therapy settings, BAT therapy not only enhances pulmonary vascular compliance and right ventricular function but also contributes to improved patient quality of life. This precision-driven approach positions BAT as a transformative intervention in secondary PH management.

### ALTERNATIVE EMBODIMENTS

### Alternative Anatomical Targets for Baroreceptor Activation

In an alternative embodiment, baroreceptor activation may be achieved by stimulating anatomical sites other than the carotid sinus. Rather than placing electrodes at the carotid sinus, an implantable or non-invasive system could be designed to target baroreceptors in the aortic arch or vagus nerve. The aortic arch contains a high density of baroreceptors, and direct stimulation of this region may produce comparable autonomic modulation effects, leading to reduced pulmonary artery pressure and improved cardiovascular function. Additionally, vagus nerve stimulation (VNS), whether via implantable electrodes or external transcutaneous stimulation, may serve as an alternative method to modulate sympathetic and parasympathetic activity, achieving pulmonary vascular resistance reduction without directly engaging the carotid baroreceptors. This embodiment may provide greater flexibility in device placement while maintaining the core objective of autonomic modulation to treat secondary PH.

### Alternative Neuromodulation Techniques

Another alternative embodiment involves using non-electrical neuromodulation techniques instead of traditional electrical stimulation. Instead of employing an IPG to deliver electrical impulses, this embodiment may utilize ultrasound-based neuromodulation, such as high-intensity focused ultrasound, to stimulate deep neural structures non-invasively. By using ultrasound waves to activate baroreceptor pathways, this approach eliminates the need for implanted electrodes while still achieving autonomic tone modulation.

Further alternative embodiments explore additional neuromodulation modalities that could achieve similar therapeutic effects. One such approach is magnetic stimulation, where external magnetic fields modulate neural activity within baroreflex pathways to regulate autonomic function. Another promising technique is optogenetic neuromodulation, in which genetically modified light-sensitive ion channels respond to light stimuli to regulate neural activity, offering a precise and potentially reversible method of autonomic modulation. Mechanical stimulation is also under investigation, involving the application of targeted mechanical forces-such as vibration, pressure, or stretch-to activate baroreceptors or associated neural pathways, thereby influencing autonomic regulation.

These alternative neuromodulation techniques offer potentially comparable reductions in sympathetic nervous system activity and pulmonary artery pressure while presenting novel approaches to secondary PH treatment.

### Non-Invasive or Minimally Invasive Baroreflex Activation

Another alternative embodiment focuses on minimizing surgical intervention by using non-invasive or minimally invasive baroreflex activation techniques.

Instead of surgically implanting electrodes on the carotid sinus, this embodiment may employ transcutaneous baroreceptor stimulation, where surface electrodes are placed on the skin over baroreceptor-rich areas, such as the neck or chest. Low-frequency electrical stimulation could then be used to activate baroreceptor pathways without the need for surgical implantation.

Beyond transcutaneous stimulation, other non-invasive embodiments provide alternative methods for delivering baroreflex activation. One such approach involves wearable neuromodulation devices, such as smart patches or bioelectronic textiles, which offer continuous stimulation in a non-intrusive manner. Another method utilizes vibrational stimulation bands, which apply mechanical, electrical, or vibratory stimuli to regulate autonomic function and improve pulmonary vascular resistance.

A further technique is the use of a pressure collar, as described in commonly-owned U.S. Publication No. 2023/0355170. This system includes a collar device designed to be worn around the user's neck and configured for releasable connection to at least one pressure (or flow) generator. The generator can be disconnected when necessary, enhancing the system's adaptability and convenience. This pressure collar delivers baroreflex activation by precisely applying pressure or flow stimuli, which are tailored to meet the user's specific physiological needs.

These approaches provide a patient-friendly alternative to surgically implanted BAT systems while offering therapeutic effects on pulmonary vascular resistance.

### Automated Sensor-Driven Drug Delivery for Pulmonary Hypertension Management

In addition to baroreceptor stimulation, an automated, sensor-driven drug delivery system may be incorporated to further enhance the management of secondary PH. This hybrid approach integrates neuromodulation with targeted pharmacologic therapy, using pressure sensors to continuously monitor pulmonary artery pressure and dynamically adjust drug administration in response to hemodynamic changes.

This system may include advanced drug delivery mechanisms—both implantable and external—that work in tandem with BAT to optimize pulmonary vascular resistance. One such mechanism involves implantable smart drug delivery devices, which automatically administer vasodilators when elevated pulmonary artery pressure is detected. Additionally, microfluidic infusion pumps may be integrated to dynamically adjust medication dosages in real time, ensuring precise pharmacologic modulation of pulmonary hemodynamics while complementing BAT-induced autonomic adjustments. Externally-worn drug delivery devices, such as wearable infusion pumps or transdermal delivery systems, may also be employed to provide flexible, non-invasive means of sustained or responsive medication delivery. These external systems can be synchronized with physiologic sensors or BAT feedback loops to enhance treatment precision without the need for surgical implantation.

By leveraging sensor-driven feedback loops, this dual-modality approach provides both neural and pharmacologic modulation, enhancing the therapeutic impact on pulmonary vascular function. Through automated drug titration, in conjunction with baroreceptor stimulation, this system delivers a personalized, responsive, and multifaceted treatment strategy for secondary PH management. By combining autonomic modulation with targeted pharmacotherapy, this approach offers a comprehensive and synergistic method for reducing pulmonary artery pressure, improving right ventricular function, and optimizing long-term cardiovascular stability.

### AI-Driven Closed-Loop Hypertension Management

Another alternative embodiment involves the implementation of AI-driven closed-loop therapy to optimize the management of secondary PH. Instead of adjusting BAT stimulation intensity in real time through a traditional closed-loop system, this approach leverages cloud-based machine learning algorithms, for example, to analyze patient-specific hemodynamic data and generate individualized therapy recommendations based on historical trends, patient-specific responses, and real-time physiological feedback.

This embodiment integrates remote patient monitoring using wireless pulmonary pressure sensors that continuously or periodically transmit hemodynamic data to a central AI system. By leveraging machine learning techniques, the AI system processes extensive patient-specific data to train predictive models that assess pulmonary vascular dynamics and autonomic function. These models enable proactive intervention, allowing for precise therapy adjustments before hemodynamic deterioration occurs. The AI-driven recommendations may include modifications to BAT parameters, medication titration, lifestyle adjustments, or alternative neuromodulation strategies based on an individual's evolving physiological profile.

By analyzing pressure data collected under different physiological conditions, such as at rest, during exercise, and under ambulatory movement, the AI system can refine therapy settings to ensure optimal outcomes across diverse activity states. This adaptive learning capability enhances the efficacy of BAT, enabling dynamic personalization of therapy that evolves with the patient's condition.

Furthermore, this AI-driven approach presents a less invasive, data-driven alternative to traditional implantable BAT devices by utilizing predictive analytics and cloud-based control. Through the integration of advanced AI analytics, remote monitoring, and real-time predictive modeling, this approach enhances secondary PH management by optimizing both autonomic and pharmacologic interventions.

By anticipating therapeutic needs and improving adaptive control mechanisms, this system ensures more precise, personalized, and proactive hypertension management, ultimately improving patient outcomes while reducing the burden of invasive procedures.

### Alternative Feedback Control Mechanisms

An additional embodiment may modify the feedback control architecture of BAT by introducing non-linear adaptive control algorithms to enhance secondary PH management. Instead of relying solely on sensor-based feedback adjustments, this approach employs predictive control models that adjust stimulation intensity based on historical patient data and long-term hemodynamic trends. By analyzing patterns in pulmonary artery pressure fluctuations, these models anticipate physiological responses and proactively fine-tune baroreceptor stimulation, allowing for more stable and personalized therapy delivery.

Further variations of this approach may include a hybrid open-loop/closed-loop model, in which clinician-supervised AI algorithms assist in manual therapy adjustments based on patient responses and clinical assessments. This model provides greater flexibility by allowing physicians to oversee and refine therapy parameters while leveraging AI-driven insights to guide optimal stimulation intensity.

By integrating alternative feedback control mechanisms, these advancements establish a distinct regulatory framework for BAT therapy, ensuring optimized pulmonary artery pressure modulation while enhancing long-term adaptability to disease progression and individual patient needs. This refined approach to feedback control strengthens autonomic modulation strategies, contributing to improved right ventricular function, pulmonary vascular compliance, and overall cardiovascular stability in secondary PH patients.

### Alternative Patient Populations and Clinical Applications

Another alternative embodiment expands the indications for BAT beyond secondary PH caused by left ventricular failure, extending its application to a broader range of cardiovascular and autonomic dysfunction disorders. In this approach, BAT may be adapted for conditions where autonomic dysregulation contributes to disease progression, including other forms of pulmonary arterial hypertension (PAH). These may encompass WHO Group 1 (Primary PAH), WHO Group 3 (PAH associated with structural lung disease), and WHO Group 4 (Chronic thromboembolic PH-CTEPH).

WHO Group 1 (Primary PAH) is characterized by idiopathic or heritable PAH, where dysregulated vascular tone and increased sympathetic activity contribute to disease severity. WHO Group 3 (PAH associated with structural lung disease) includes patients with chronic obstructive pulmonary disease (COPD), interstitial lung disease, or hypoxia-driven PH, where BAT may help modulate pulmonary vascular resistance and improve cardiopulmonary interactions. WHO Group 4 (Chronic thromboembolic PH-CTEPH) is a form of PH caused by unresolved pulmonary emboli, where BAT could serve as an adjunct therapy to improve hemodynamic stability and vascular remodeling.

By extending BAT applications beyond secondary PH, this embodiment highlights its potential as a novel neuromodulatory therapy for a wide range of pulmonary and autonomic disorders, addressing an unmet clinical need in the management of progressive cardiovascular dysfunction.

### Hybrid Neuromodulation and Multimodal Therapy Approaches

Lastly, another alternative embodiment integrates BAT with other neuromodulation or pharmacological interventions, creating a multimodal treatment platform for optimizing autonomic function and pulmonary vascular regulation.

This hybrid approach may combine BAT with renal denervation to further reduce sympathetic activity, VNS to enhance parasympathetic function, or direct cardiac neuromodulation to optimize cardiac output and hemodynamic stability. By addressing multiple autonomic pathways, this strategy enhances the overall therapeutic impact on secondary PH management.

Further alternative embodiments may integrate neuromodulation with gene therapy or pharmaceutical treatments, leveraging synergistic effects to modulate pulmonary artery pressure and vascular resistance more effectively. The combination of BAT with targeted pharmacologic or genetic interventions offers a personalized, adaptive approach to autonomic dysfunction, ensuring greater flexibility in treatment strategies.

By incorporating multiple therapeutic modalities, this embodiment provides a comprehensive, integrative solution for managing pulmonary vascular resistance and autonomic dysfunction. This synergistic approach enhances long-term cardiovascular stability, expanding the clinical applications of BAT therapy beyond standalone neuromodulation.

### Conclusion

By integrating BAT with physiological monitoring, control algorithms, and/or individualized therapy optimization, the present systems and methods represent a transformative approach to the management of secondary PH. The combination of pressure monitoring, adaptive therapy titration, and feedback-driven control can ensure that patients receive precise, effective, and personalized treatment tailored to their unique hemodynamic needs.

The present systems and methods have the potential to improve survival rates, enhance quality of life, and significantly reduce the clinical burden of secondary PH. By leveraging advanced neuromodulation technology, sensor-driven feedback mechanisms, and adaptive patient-centered therapy, the systems and methods establish a novel, clinically significant therapeutic strategy for the effective management of secondary PH, providing long-term benefits for patients with both acute and chronic forms of the disease.

The above Detailed Description includes references to the accompanying drawings, which form a part of the Detailed Description. The Detailed Description should be read with reference to the drawings. The drawings show, by way of illustration, specific embodiments in which the present systems and methods can be practiced. These embodiments are also referred to herein as “examples.”

The above Detailed Description is intended to be illustrative and not restrictive. For example, the above-described examples (or one or more features or components thereof) can be used in combination with each other. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:

In Example 1, a method for treating secondary PH in a patient comprises applying BAT through an implantable or external stimulation device to modulate autonomic nervous system activity and reduce pulmonary vascular resistance; measuring pulmonary arterial pressure using a pressure sensor; and adjusting one or more BAT parameters in response to the measured pressure.

In Example 2, the method of Example 1 is optionally configured such that baroreflex activation therapy is applied to carotid sinus baroreceptors to decrease sympathetic nervous system activity and increase parasympathetic nervous system activity.

In Example 3, the method of any one of Examples 1 or 2 is optionally configured such that measuring pulmonary arterial pressure includes using an invasive pressure sensor.

In Example 4, the method of any one of Examples 1 or 2 is optionally configured such that measuring pulmonary arterial pressure includes using a non-invasive pressure sensor.

In Example 5, the method of any one of Examples 1-4 is optionally configured such that baroreflex activation therapy is applied intermittently based on hemodynamic monitoring.

In Example 6, the method of any one of Examples 1-5 optionally further comprises determining a patient's eligibility for baroreflex activation therapy based on measured pulmonary arterial pressure.

In Example 7, the method of any one of Examples 1-6 is optionally configured such that BAT parameters are adjusted based on patient-reported outcomes, measured through standardized questionnaires assessing exercise tolerance and quality of life.

In Example 8, the method of any one of Examples 1-7 is optionally configured such that the BAT stimulation device is programmed to apply different stimulation patterns during periods of rest and exertion, based on measured pulmonary arterial pressure fluctuations.

In Example 9, the method of any one of Examples 1-7 is optionally configured such that the BAT stimulation device is programmed to apply different stimulation patterns based on patient activity level.

In Example 10, the method of any one of Examples 1-9 is optionally configured such that BAT parameters are adjusted based on a dose-response curve, correlating measured pulmonary arterial pressure data with one or more baroreflex activation therapy parameters.

In Example 11, the method of any one of Examples 1-10 is optionally configured such that therapy effectiveness is assessed by correlating one or more baroreflex activation therapy parameters with measured pulmonary arterial pressure trends over a predetermined treatment period.

In Example 12, a system for treating secondary PH in a patient comprises an implantable BAT device configured to deliver electrical stimulation to baroreceptors; and a pressure monitoring module configured to measure pulmonary arterial pressure and provide feedback to adjust BAT parameters.

In Example 13, the system of Example 12 is optionally configured such that the pressure monitoring module comprises an invasive pressure sensor.

In Example 14, the system of Example 12 is optionally configured such that the pressure monitoring module comprises a non-invasive pressure sensor.

In Example 15, the system of any one of Examples 12-14 optionally further comprises a closed-loop control mechanism configured to adjust BAT parameters in response to measured pulmonary arterial pressure data.

In Example 16, the system of Example 15 is optionally configured such that the closed-loop control mechanism utilizes artificial intelligence algorithms to optimize therapy delivery based on historical patient data.

In Example 17, the system of Example 15 is optionally configured such that the closed-loop control mechanism titrates BAT based on historical patient data, measured pulmonary pressure trends, or predicted pulmonary pressure trends.

In Example 18, the system of any one of Examples 12-14 optionally further comprises an open-loop control mechanism, allowing manual adjustments of BAT parameters.

In Example 19, the system of Example 18 is optionally configured such that manual adjustment is based on pulmonary arterial pressure data or a surrogate parameter.

In Example 20, the system of Example 18 is optionally configured such that manual adjustment is based on the patient's qualitative status or preferences.

In Example 21, the system of Example 18 is optionally configured such that the open-loop control mechanism includes a user interface, allowing clinicians or the patient to adjust BAT parameters.

In Example 22, the system of Example 21 is optionally configured such that the user interface requires clinician oversight for certain therapy adjustments, wherein therapy modifications initiated by the patient are subject to predefined safety thresholds. If these thresholds are exceeded, the modifications are transmitted to a clinician for review and approval before implementation.

In Example 23, the system of any one of Examples 12-22 is optionally configured such that the pressure monitoring module comprises a multi-sensor array capable of measuring systemic arterial pressure, pulmonary arterial pressure, and right atrial pressure.

In Example 24, the system of Example 23 is optionally configured such that the pressure monitoring module measures systemic arterial pressure, pulmonary arterial pressure, or right atrial pressure under varying physiological conditions, including rest, exercise, and ambulatory states.

In Example 25, the system of Example 24, wherein the collected data is used to optimize BAT parameters based on patient-specific hemodynamic responses across different activity levels.

In Example 26, the system of any one of Examples 12-25 optionally further comprises an alert system that provides feedback regarding the patient's therapy status and indicates potential adjustments needed based on sensor data.

In Example 27, the system of any one of Examples 12-26 optionally further comprises a data storage module configured to record pulmonary arterial pressure measurements and corresponding BAT settings.

In Example 28, the system of any one of Examples 12-27 is optionally configured such that BAT parameters are stored in a cloud-based database, enabling remote monitoring and collaborative clinician decision-making.

### Definitions and Interpretations

Throughout this patent document, specific terms are used to refer to particular features, components, and method steps. As one skilled in the art will recognize, different terminology may be used to describe the same or similar elements. The terminology used herein is not intended to limit the scope of the invention to any particular nomenclature. Instead, the terms should be understood in accordance with their function within the systems and methods described.

Unless explicitly defined otherwise within this document, the following definitions shall apply. The terms “a,” “an,” and “the” refer to one or more than one, irrespective of whether the phrase “at least one” or “one or more” is used in other contexts. The term “or” should be interpreted as a nonexclusive disjunction, meaning that “A or B” includes (i) A but not B, (ii) B but not A, and (iii) both A and B.

All numerical values provided in this disclosure should be understood as approximate values, whether explicitly stated or not, and are assumed to be modified by the term “about.” The term “about” encompasses numerical variations that are considered functionally equivalent by those skilled in the art. This may include rounding to the nearest significant figure or values within an equivalent range. Additionally, when numerical ranges are provided, the endpoints are inclusive of all intermediate values and subranges. For example, the range 1 to 4 includes 1, 1.5, 1.75, 2, 2.3, 2.6, 2.9, etc., as well as subranges such as 1 to 1.5, 1 to 2, 1 to 3, 2 to 3.5, 2 to 4, 3 to 4, etc.

### Scope and Claim Construction

The scope of the present systems and methods should be determined with reference to the appended claims, along with the full scope of equivalents to which such claims are entitled. In the claims, the term “including” is to be interpreted as the plain-English equivalents of “comprising” and “wherein.” The use of “including” and “comprising” in a claim should be understood as open-ended terminology, meaning that a system or method that includes additional features or components beyond those listed still falls within the scope of the claim.

### Abstract Interpretation

The Abstract of this application is provided solely for the purpose of enabling a quick understanding of the technical disclosure. It is submitted with the express understanding that it will not be used to interpret or limit the scope or meaning of the claims. The present systems and methods should be construed based on the full scope of the Detailed Description and claims, rather than any limitations inferred from the Abstract.

1. A method for treating secondary pulmonary hypertension in
a patient, comprising:
applying electrical baroreflex activation therapy (BAT) using a stimulation device configured to stimulate a baroreceptor to modulate autonomic nervous system activity;
measuring pulmonary arterial pressure using a pulmonary arterial pressure sensor, and
arterial pressure using a closed-loop control algorithm in response to fluctuations in the measured pulmonary arterial pressure, thereby dynamically titrating the at least one stimulation parameter so as to reduce pulmonary vascular resistance and right ventricular afterload.

2. The method of claim 1, wherein the baroreceptor comprises a carotid sinus baroreceptor.

3. The method of claim 1, wherein the baroreceptor comprises an aortic arch baroreceptor.

4. The method of claim 1, wherein the stimulation device comprises an implantable stimulation device.

5. The method of claim 1, wherein the stimulation device comprises an external stimulation device.

6. The method of claim 1, wherein adjusting comprises operating in a closed-loop mode in which stimulation intensity is automatically modulated based on pulmonary artery pressure fluctuations measured during rest, exercise, or ambulatory movement.

7. The method of claim 1, wherein stimulation parameters are adjusted differently during rest and exercise states based on measured pulmonary artery pressure fluctuations.

8. The method of claim 1, wherein adjusting comprises referencing a dose-response relationship correlating pulmonary artery pressure reduction with stimulation intensity.

9. The method of claim 8, wherein stimulation is selected within a linear region of a sigmoidal dose-response curve to avoid overstimulation.

10. The method of claim 1, wherein the method further comprises determining patient eligibility for BAT based on a pulmonary arterial pressure remaining above a predefined threshold despite administration of at least one pulmonary hypertension medication.

11. A system for treating secondary pulmonary hypertension in
a patient, comprising:
a stimulation device configured to deliver electrical baroreflex activation therapy to a baroreceptor;
a pulmonary arterial pressure sensor configured to obtain pulmonary arterial pressure measurements;
at least one additional physiological sensor configured to measure one or more of: right atrial pressure, systemic arterial pressure, heart rate variability, activity level, or respiratory cycle dynamics; and
a processor configured to: (i) receive data from the pulmonary arterial pressure sensor and the additional physiological sensor; and (ii) implement a closed-loop control algorithm to dynamically regulate stimulation intensity based on the received data.

12. The system of claim 11, wherein the stimulation device comprises an implantable pulse generator.

13. The system of claim 11, wherein the stimulation device comprises an external stimulation device.

14. The system of claim 11, wherein the processor computes a deviation of measured pulmonary arterial pressure from a target range and adjusts stimulation intensity based on the deviation.

15. The system of claim 11, wherein the processor integrates heart rate variability, activity level, or respiratory cycle dynamics with measured pulmonary arterial pressure when regulating stimulation intensity.

16. The system of claim 11, further comprising an intermittent pressure monitoring module configured to reduce sensor fatigue.

17. The system of claim 11, wherein the processor utilizes a stored dose-response model correlating pulmonary artery pressure reduction with stimulation intensity.

18. The system of claim 11, wherein the processor utilizes historical pulmonary arterial pressure data to predict future pulmonary arterial pressure and adjusts stimulation intensity before the predicted pulmonary arterial pressure change occurs.

19. The system of claim 11, further comprising a user interface requiring clinician approval for therapy modifications exceeding predefined safety thresholds.

20. The system of claim 11, further comprising a memory device storing pulmonary arterial pressure measurements and corresponding stimulation parameters.
